(19) United States
(12) Patent Application Publication
(10) Pub. No.: US 2017/0073829 A1
(43) Pub. Date: Mar. 16, 2017

(54) METHOD FOR REDUCING THE FORMATION OF FLUOROCARBONS IN MOLTEN SALT ELECTROLYSIS

(71) Applicant: SIEMENS AKTIENGESELLSCHAFT, Munchen (DE)

(72) Inventors: Bernd FRIEDRICH, Aachen (DE); Marc HANEBUTH, Nuremburg (DE); Alexander TREMEL, Erlangen (DE); Hanno VOGEL, Monheim (DE)

(73) Assignee: SIEMENS AKTIENGESELLSCHAFT, Munchen (DE)

(21) Appl. No.: 14/852,933

(22) Filed: Sep. 14, 2015

(51) Int. Cl. C25C 7/06; G01N 33/00; C25C 3/00

(52) U.S. Cl. CPC C25C 7/06; C25C 3/00; G01N 33/0049

(57) ABSTRACT

A sensor is provided for measuring the concentration of a fluorocarbon in the offgas during molten salt electrolysis of metal compounds. The measurement takes place at time intervals of less than 10 seconds and a controller initiates reduction in an electrolysis voltage if a fluorocarbon limit value of 25 ppm is exceeded.

### CROSS REFERENCE TO RELATED APPLICATIONS

This application claims the benefit of German Application No. 10 2014 218 440.1 filed on Sep. 15, 2014 which is incorporated by reference herein in its entirety.

### BACKGROUND

Described below is a method for reducing the formation of fluorocarbons in molten salt electrolysis.

Many metals such as aluminum or also the so-called rare earth elements are recovered in pure form in a molten salt electrolysis process. Here an electrolyte, often a fluoride, is brought to melting temperature and a compound, generally an oxide, of the metal to be reduced is introduced into this molten electrolyte. There are also two electrodes, an anode which generally extends into the electrolysis bath from above and is formed of graphite, and a cathode which can be formed, e.g., of tungsten or molybdenum and projects into the electrolytic bath at another point. It is also extremely advantageous to use so-called floor cathodes which are disposed in the form of plates on the floor of the molten bath. The cations of the metal to be recovered are reduced at the cathodes and accumulate as molten metal, as the operating temperature is set such that it is above the melting point of the respective metal to be recovered. At the anode, the oxygen anions are oxidized and react with the carbon of the graphite anode to form carbon monoxide and carbon dioxide. The gaseous reaction products leave the electrolyte and pass into an offgas. If a critical anode current density is exceeded, or the electrolyte is depleted in oxygen, fluorocarbons, e.g. CF₄, may be produced which form a passivating film on the anode. The resulting breakdown of electrical conductivity is known as the anode effect.

In the publication “Die Reduktion von Treibhausgasen in der Elektrolyse” (“Reduction of greenhouse gases in electrolysis”) by Martin Inert et al. in ERZMETALL 55 (2002), No. 8, the treatment of such anode effects in aluminum production is described. This paper shows different strategies for the so-called termination of such anode effects. It describes how the success rate can be increased from 60 to 90% by the so-called termination strategies for terminating anode effects in molten salt electrolysis. However, the problem with the technique described is that it is first necessary to wait until anode effects occur and then attempt to terminate them by suitable methods.

### SUMMARY

Described below is a method which is suitable for preventing anode effects from occurring.

The method reduces the formation of fluorocarbons in molten salt electrolysis of metal compounds using a sensor provided for measuring the concentration of a fluorocarbon in the offgas. During the method, the measurement takes place at time intervals of less than 10 s and a control device initiates a reduction of an electrolysis voltage if a fluorocarbon limit value of 100 ppm (parts per million) is exceeded.

In the proposed solution, the time interval for measuring the fluorocarbons in the offgas is quite significantly reduced compared to the prior art. Here a measurement of the fluorocarbons in the offgas is performed at least every 10 s and a very low limit value of 100 ppm, 10 ppm, or even 1 ppm, is set at which countermeasures are initiated even before anode effects occur. In contrast to the state of the art described, the method described makes it possible to prevent anode effects even before they occur. For this purpose it is necessary to minimize the measurement interval for measuring the fluorocarbon concentration, a real-time measurement being ideal here. As every known measuring method currently available includes a certain dead time, only a quasi real-time measurement can come into consideration. In particular it is advantageous to set the time interval for measuring fluorocarbons to less than 2 s, or less than 1 s, or even less than 0.5 s. In the case of the currently existing technical options for measuring fluorocarbons, a time interval of 10 s provides a quasi real-time measurement. In addition, a fluorocarbon limit value of 25 ppm is assumed. It has been found that at a technically measurable low limit value of this kind, no anode effects appear. It may also be advisable to select this limit value lower, e.g. 1 ppm, in order to forestall the development of anode effects even earlier.

It is advantageous, unlike as described in the prior art, to measure the fluorocarbon concentration at a plurality of points above the melt surface, as anode effects may occur spontaneously at very different locations. In addition, the measurement must be performed as close as possible to the melt surface, 50 cm being advantageous here. The closer the measuring probe or sensor is to the surface, the quicker will be the reaction to measured fluorocarbon, and countermeasures to prevent anode effects can be initiated.

In the following, a cell is to be understood as meaning a sealed unit which contains a coherent electrolyte and in which an electrolytic reaction takes place. It is advantageous to provide a single measuring point for each cell. In the case of larger cells which are operated at an electrolysis current density of 10 kA or more it is advantageous to provide a plurality of measuring points for each cell. In this case it is particularly advantageous to provide at least two measuring points per cell, but no more than one measuring point per anode. An anode is here an electrode unit which projects into the electrolyte and can be moved or replaced independently of the other anodes in the cell.

An anode effect can likewise be prevented even before it occurs by submerging the anode in the melt and increasing the oxide dosing.

Further embodiments and other features of the invention will be explained with reference to the accompanying drawings. These are purely exemplary embodiments which do not limit the scope of protection sought.

### BRIEF DESCRIPTION OF THE DRAWINGS

These and other aspects and advantages will become more apparent and more readily appreciated from the following description of the exemplary embodiments, taken in conjunction with the accompanying drawings, of which:

FIG. 1 is a graph of a measurement curve illustrating anode effects as a function of the current and of the electrolysis voltage,

FIG. 2 is a schematic block diagram of a molten salt electrolysis apparatus having an apparatus for preventing anode effects.

### DETAILED DESCRIPTION OF THE PREFERRED EMBODIMENT

Reference will now be made in detail to the preferred embodiments, examples of which are illustrated in the accompanying drawings, wherein like reference numerals refer to like elements throughout.

The technical recovery of metals by molten salt electrolysis will now be described in detail using so-called rare earth metals as an example. Similar methods are also used in aluminum production. The electrolytic process used here is composed of a plurality of process steps. First an electrolyte generally contains fluorides. The main components here are lithium fluoride and a rare earth fluoride. The rare earth fluoride must contain the rare earth which is to be produced electrolytically in metal form. Admixtures of other alkali or alkaline earth fluorides are likewise possible. The electrolyte is adjusted in respect of the melting point, vapor pressure and conductivity as well as solubility for the electrolytic raw material and density. The electrolyte is contained in a suitable crucible which generally contains carbon. However, it can also be formed of other stable materials. In the currently used 3 kA and 4 kA technology, the crucible has a circular shape and a diameter of less than 1 m. The crucible is again lined with refractory material and surrounded by a steel wall. In larger cells, which are supplied with current up to 30 kA, the cell is typically of rectangular design and several meters long. Anodes 12 (cf. FIG. 2) project perpendicularly from above into the electrolytic bath and into the electrolyte. The anodes 12 may be graphite or suitable carbon material and can be inserted as blocks or as circular segments. A cathode may be tungsten or molybdenum and has different designs. The cathodes can be rod-shaped and immersed in the electrolyte between the anodes 12. Another embodiment as illustrated in FIG. 2 and used in rare earth electrolysis is a so-called floor cathode 14. Here the rod cathode is not lowered perpendicularly from above in the usual way; instead, these floor cathodes 14 are disposed on the floor of the electrolytic cell in the form of tungsten plates. In this floor variant, the floor cathode 14 is covered by the metal produced. The molten rare earth metal then likewise acts as a cathode.

Rare earth compounds are charged into the electrolyte. In this state this is termed the melt 24 in FIG. 2. The rare earth compounds may be rare earth oxides, but carbonates or other suitable rare earth compounds can also be supplied. Rare earth oxides are in many cases the end products of the separation process immediately preceding electrolysis, so that these are suitable as electrolytic material. This compound must also dissolve in the electrolyte, wherein rare earth fluorides only dissolve a small percentage of rare earth oxides. The dissolved rare earth oxides are present as ions in the electrolyte. At the floor anode 14, the rare earth cations are reduced and accumulate as molten metal, as the operating temperature is above the melting point of the respective rare earth metal. At the anode, the oxygen ions are oxidized and react with the carbon of the graphite at the graphite anode 11 to form carbon dioxide and carbon monoxide. The gaseous reaction products leave the electrolyte and enter the offgas. If a critical anode current density is exceeded, or the electrolyte is oxygen depleted, fluorocarbons, particularly CF₄ or C₂F₆, may also be produced which form a passivating film. The resulting breakdown of the electrical conductivity is known as the anode effect. Prior to the occurrence of the anode effect, resulting in the collapse of electrolysis, fluorocarbons in the form of CF₄ and in traces C₂F₆ are emitted, as the lack of oxygen anions results in oxidation of fluorine anions. Herein lies a problem in the process control of molten salt electrolysis per se, as the fluorocarbons described are extremely powerful greenhouse gases whose effect is many thousand times greater than carbon dioxide.

If anode effects have already occurred, they can only be overcome with great difficulty. The related art teaches a number of methods of doing so. However, during the time in which the anode effects are being terminated, a not inconsiderable amount of fluorocarbons escapes and enters the atmosphere. It is therefore advantageous to control the molten salt electrolysis process such that suitable action is taken even before these anode effects occur, so that these anode effects fail to materialize.

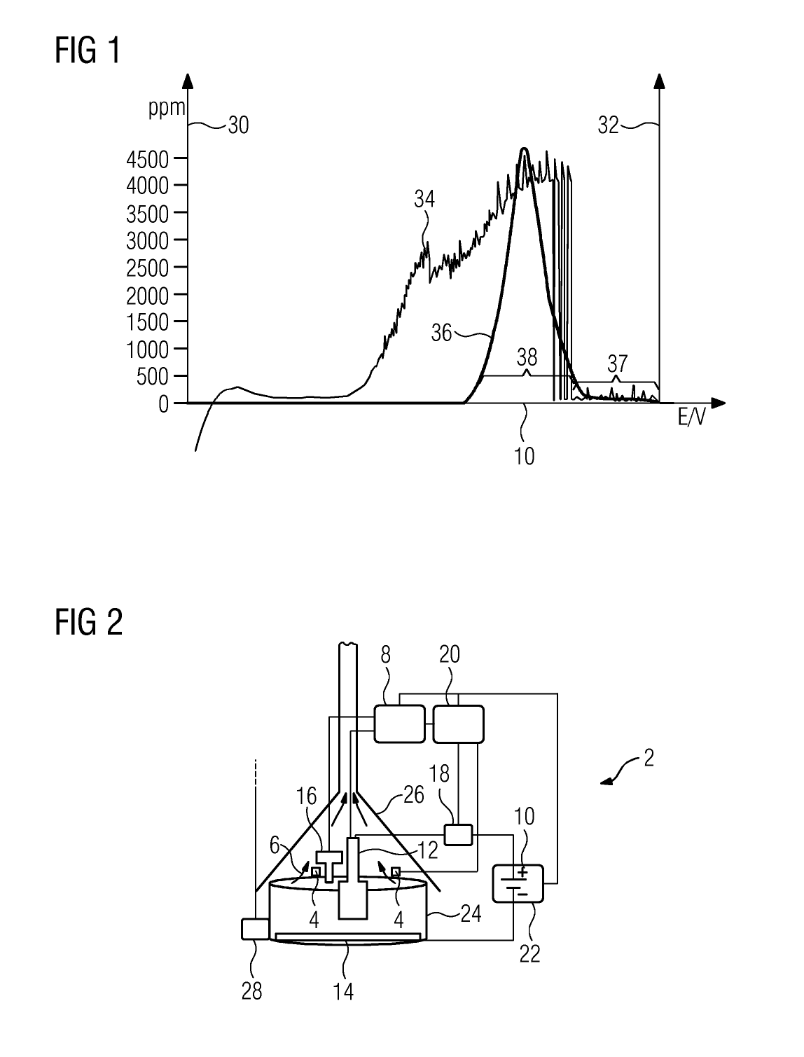

FIG. 1 shows a two-in-one graph, wherein the graph has a left-hand Y-axis 30 in which the fluorocarbon concentration is plotted and a right-hand Y-axis 32 in which the intensity of the current flowing through the molten salt electrolysis bath is qualitatively plotted. The electrolysis voltage 10 is plotted on the X-axis. The curve 36 therefore shows the fluorocarbon concentration as a function of the electrolysis voltage. The curve 34 shows the current intensity likewise as a function of the electrolysis voltage. It can be seen from FIG. 1 that the current intensity increases continuously up to a region 35 of so-called fluctuating anode effects in which the current intensity rises and falls very suddenly and very strongly, until finally the current intensity has dropped to approximately 0 in the region 37. This region 37 is termed complete anode effect.

However, the curve 36 which shows the concentration of fluorocarbons as a function of the electrolysis voltage 10 teaches that the highest concentration of fluorocarbons occurs even before the anode effects. This is particularly the case in the region 38. FIG. 1, in particular the curves 34 and 36 taken together, teaches that when classical anode effects occur in the regions 35 and 37, the majority of the environmentally harmful fluorocarbons have already found their way into the offgas and therefore into the environment. However, the occurrence of the fluorocarbons is not evident from the current/voltage curve 34 alone. It is therefore advantageous to take appropriate action well before the increase in fluorocarbons occurs in the curve 36. For this purpose, fluorocarbon concentration measurements are performed at maximally short time intervals, possibly of less than 10 s. These measurements can be performed e.g. photoacoustically or by infrared technology. It is advantageous to carry out quasi-continuous measurement. The measurement should take place as quickly as technically and economically possible, as the rise of the curve 36 can proceed very rapidly in the region 38. If a particular fluorocarbon concentration limit, e.g. 25 ppm, is exceeded, the electrolysis voltage 10, or also the electrolysis current intensity if the electrolytic cell is being operated under current intensity control, is reduced as a countermeasure. This reduction takes place well before the occurrence of anode effects as illustrated in the region 35 or 37, for example. Anode effects and an increased emission of fluorocarbons are prevented by this action.

FIG. 2 schematically illustrates an apparatus for molten salt electrolysis 2. The offgas analysis described can in principle be carried out using commonly employed gas analysis methods from the prior art, e.g., gas chromatographs. Advantageous, however, is an infrared spectrometer, e.g. a Fourier transform infrared spectrometer, which continuously records even small concentrations of all the important gas components. Alternatively, it may be particularly advantageous to use a gas analysis method based on the photoacoustic effect, as it provides particularly simple and robust sensors 10 that are sensitive to IR-active molecules. The CO and CO₂ and CₓF_{y} compounds, particularly CF₄ and C₂F₆, necessary for the early detection of anode effects are IR-active and can therefore be measured online at short measurement intervals of just few seconds, in particular less than 10 s, using an infrared spectrometer or a photoacoustic gas sensor.

All measured values are recorded by a central data logger 20 which graphically displays the measured values. This data logger is connected to a controller 20 which has its own control algorithm. This controller 8 controls, as a function of the measured offgas concentration, the current I (axis 32 in FIG. 1) or the electrolysis voltage 10 (X-axis of FIG. 1) and optionally also an oxide dosing device 16 and possibly also a position of the anode 12 with respect to its vertical position.

Depending on the state of the raw material, the dosing of the electrolyzing compound is accordingly implemented as a powder doser or any other commonly used form from the related art. In general, defined dosing by mass and time takes place. The dosing can proceed either continuously or discontinuously. The effective mass flow of the dosing must be selected high enough to ensure that the electrolyte is not depleted in the respective raw material, but at the same time must not be so high that no supersaturation of the compound e.g. of rare earth oxide in the electrolyte takes place, as otherwise silting-up of the molten salt hydrolysis 2 or of the melt 24 may take place. The necessary mass flow can be determined from the faradaic current in combination with the current efficiency. Alternatively or supportively, a conductivity measurement of the electrolyte or of the melt 24 or an oxygen measurement in the electrolyte can be carried out for this purpose.

Another possibility for counteracting an anode effect is to adjust the height of the electrode. Increasingly immersing the anode results in a larger contact surface area with the electrolyte and therefore a lower current density. The controller 8 can therefore also react to the offgas concentration by adjusting the height of the anode.

By the countermeasures described, which can be initiated if even small amounts of fluorocarbon compounds are detected, the current density at the anode is reduced and/or the oxygen concentration in the melt 24 is increased so that a full anode effect, as can be seen in the region 37 according to FIG. 1, can be counteracted in good time. The effectiveness of the countermeasures can be monitored directly by the falls in the CF concentration.

It is also advantageous that the controller 8 limits the voltage rise and therefore simultaneously limits the current density at the anode 12 if predefined limit values are exceeded. In addition, an oxide dosing device 16 can be controlled in order to increase the oxygen ion concentration in the electrolyte or more specifically the melt 24.

The molten salt electrolysis apparatus shown in FIG. 2 includes the already described components and the anode 12 is also connected to a shunt resistor 18 and a rectifier 22. The sensor 4 used for measuring the CF concentration must be disposed as closely as possible above the surface of the melt 24. It must be as close to the surface as is technically feasible in terms of process control and, in particular, temperature. The closer the sensor 4 is placed to the surface, the earlier fluorocarbons occurring can be detected. FIG. 2 shows two sensors 4, but generally the use of a plurality of sensors 4 may be advantageous in large installations.

In FIG. 2, a so-called floor cathode 14 made of tungsten is used on which the melted rare earth metal is deposited and which in turn acts as a cathode. The molten reduced rare earth metal can be removed at a metal tap 28.

A description has been provided with particular reference to preferred embodiments thereof and examples, but it will be understood that variations and modifications can be effected within the spirit and scope of the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

What is claimed is:

1. A method for reducing formation of fluorocarbons in molten salt electrolysis of metal compounds, comprising:
measuring, by a sensor, concentration of a fluorocarbon in offgas at time intervals of less than 10 seconds; and
initiating, by a controller, a reduction in at least one of an electrolysis voltage and an electrolysis current density at an anode, when a fluorocarbon limit value of not more than 100 ppm is exceeded.

2. The method as claimed in claim 1, wherein said measuring of the fluorocarbon concentration takes place in intervals of less than 2 seconds.

3. The method as claimed in claim 2, wherein said measuring of the fluorocarbon concentration takes place in intervals of less than one second.

4. The method as claimed in claim 2, wherein said measuring of the fluorocarbon concentration takes place in intervals of less than 0.5 second.

5. The method as claimed in claim 1, wherein the fluorocarbon limit value is 10 ppm.

6. The method as claimed in claim 1, wherein the fluorocarbon limit value is 1 ppm.

7. The method as claimed in claim 1, wherein said measuring of the concentration of the fluorocarbon occurs at a plurality of points over a melt surface.

8. The method as claimed in claim 7, wherein one measuring point for said measuring of the concentration of the fluorocarbon is provided per 10 kA current intensity present in an electrolysis system.

9. The method as claimed in claim 7, wherein one measuring point for said measuring of the concentration of the fluorocarbon is provided per 2 kA current intensity present in an electrolysis system.

10. The method as claimed in claim 7, wherein one measuring point for said measuring of the concentration of the fluorocarbon is provided per 1 kA current intensity present in an electrolysis system.

11. The method as claimed in claim 1, wherein said measuring takes place at a height of less than 50 cm above a melt surface.

12. The method as claimed in claim 1, wherein said measuring takes place at a height of less than 25 cm above a melt surface.

13. The method as claimed in claim 1, further comprising varying an immersion depth of the anode in reaction to an increase in the concentration of the fluorocarbon.

14. The method as claimed in claim 1, further comprising increasing oxide ion dosing in reaction to an increase in the concentration of the fluorocarbon.
